(19) United States
(12) Patent Application Publication
(10) Pub. No.: US 2019/0119657 A1
(43) Pub. Date: Apr. 25, 2019

(54) GENUS KOMAGATAEIBACTER RECOMBINANT MICROORGANISM, METHOD OF PRODUCING CELLULOSE USING THE SAME, AND METHOD OF PRODUCING THE MICROORGANISM

(71) Applicant: Samsung Electronics Co., Ltd., Suwon-si (KR)

(72) Inventors: Jaeyoung Kim, Suwon-si (KR); Hongsoon Rhee, Suwon-si (KR); Jinhwan Park, Suwon-si (KR)

(21) Appl. No.: 16/028,145

(22) Filed: Jul. 5, 2018

(30) Foreign Application Priority Data
Oct. 19, 2017 (KR) 10-2017-0135865

(51) Int. Cl. C12N 9/12; C12N 15/74; C12N 15/69; C12P 19/04

(52) U.S. Cl. CPC C12N 9/1205; C12Y 207/01002; C12P 19/04; C12N 15/69; C12N 15/74

(57) ABSTRACT

Provided is genus Komagataeibacter microorganism having enhanced cellulose productivity and yield, a method of producing cellulose using the same, and a method of producing the microorganism.

### CROSS-REFERENCE TO RELATED APPLICATION

This application claims the benefit of Korean Patent Application No. 10-2017-0135865, filed on Oct. 19, 2017, in the Korean Intellectual Property Office, the entire disclosure of which is hereby incorporated by reference.

### INCORPORATION BY REFERENCE OF MATERIAL SUBMITTED ELECTRONICALLY

Incorporated by reference in its entirety herein is a computer-readable nucleotide/amino acid sequence listing submitted concurrently herewith and identified as follows: One 84,745 Byte ASCII (Text) file named “739059_ST25.TXT”, created on Jun. 8, 2018.

### BACKGROUND

### 1. Field

The present disclosure relates to a recombinant genus Komagataeibacter microorganism, a method of producing cellulose using the same, and a method of producing the microorganism.

### 2. Description of the Related Art

Plant-based celluloses are present in large numbers and are inexpensive, and interest therein is increasing in recent years. However, due to the presence of lignin and hemicelluloses among lignocellulosic biomass, a complicated process is required to prepare plant-based celluloses for medical purposes. Bacterial cellulose is an insoluble extracellular polysaccharide produced by bacteria, such as bacteria of the genus Acetobacter. Bacterial cellulose exists as a primary structure, β-1,4 glucan, which forms a network structure of fibril bundles. Bacterial cellulose is a highly pure form of cellulose with a fine nano-scale structure. Additionally, bacterial cellulose has excellent physicochemical characteristics including high mechanical tensile strength, high purity, high biodegradability, high water-holding capacity, and high heat resistance. Due to these characteristics, bacterial cellulose has been developed for use in a variety of applications, such as cosmetics, medical products, dietary fibers, audio speaker diaphragms, functional films, and the like.

Therefore, there is a need to develop new microorganisms and methods to increase the production of microbial cellulose. This invention provides such microorganisms and methods.

### SUMMARY

Provided is a recombinant microorganism of genus Komagataeibacter including a genetic modification that increases activity of a cellulose synthase.

Another aspect provides a method of producing cellulose using the microorganism.

Another aspect provides a method of producing the microorganism.

### BRIEF DESCRIPTION OF THE DRAWINGS

These and/or other aspects will become apparent and more readily appreciated from the following description of the embodiments, taken in conjunction with the accompanying drawings in which:

FIG. 1 illustrates the structure of a vector pIN04 for use in inserting four genes into a microorganism; and

FIG. 2 illustrates a pIN04-galP-xanA-galU-glk vector including four genes.

### DETAILED DESCRIPTION

The terms “increase in activity” or “increased activity”, or like terms, as used herein, refers to a detectable increase in activity level of a cell, a protein, or an enzyme, relative to the activity level of a cell, protein, or enzyme that does not have a given genetic modification (e.g., a parent cell or a native original, or “wild-type” cell, protein, or enzyme). For example, an activity of a modified or engineered cell, protein, or enzyme may be increased by about 5% or more, about 10% or more, about 15% or more, about 20% or more, about 30% or more, about 50% or more, about 60% or more, about 70% or more, or about 100% or more relative to the activity of a cell, protein, or enzyme of the same type that does not have a given modification or has not been engineered (e.g., a parent or wild-type cell, protein, or enzyme). A cell having an increased activity of a protein or enzyme may be identified by using any method known in the art.

A cell having increased activity of an enzyme or a polypeptide may be achieved by an increase in expression of a gene or polynucleotide encoding the enzyme or polypeptide, or by increasing the specific activity of the enzyme or polypeptide. As used herein, the terms “gene’ and “polynucleotide” are used synonymously to refer to a nucleic acid encoding a polypeptide, unless otherwise indicated. The increase in the expression may be achieved by introduction of a polynucleotide encoding the enzyme or the polypeptide into a cell; by otherwise increasing the copy number of a polynucleotide that encodes the enzyme or polypeptide in a cell; or by modification (mutation) of a regulatory region of the polynucleotide that increases expression of the enzyme or polypeptide. When a polynucleotide is introduced into the cell, the introduction may be a transient introduction in which the gene is not integrated into a genome, or an introduction that results in integration of the gene into the genome. The introduction may be performed, for example, by introducing a vector comprising a polynucleotide encoding the enzyme or peptide into the cell. A microorganism to be introduced with the polynucleotide may include a copy of the polynucleotide or may not include a copy of the polynucleotide prior to introduction of the exogenous polynucleotide.

The polynucleotide may be operably linked to a regulatory sequence that allows expression of the enzyme or polypeptide, for example, a promoter, a polyadenylation region, or a combination thereof. The polynucleotide may be endogenous or heterologous with respect to the microorganism into which it is inserted. As used herein, an endogenous gene refers to a polynucleotide that is present in the intrinsic genetic material of the microorganism prior to a given genetic manipulation, for instance, the native genetic material of a wild-type microorganism. As used herein, an exogenous gene refers to a polynucleotide that is introduced into a cell. The gene to be introduced may be endogenous (homologous) or heterologous with respect to a host cell into which the polynucleotide is introduced. As used herein, the term “heterologous” means “not native” or “foreign” with respect to a given species, and “homologous” or “endogenous” means “native” with respect to a given species

An “increase in the copy number” of a polynucleotide refers to any increase in copy number. For example, an increase in copy number may be caused by introduction of an exogenous polynucleotide or amplification of an endogenous polynucleotide, and may be achieved by introducing a heterologous polynucleotide that is not present in the non-engineered cell or parent cell. The introduction of a polynucleotide may be transient introduction in which the polynucleotide is not integrated into a genome thereof, or may be insertion of the polynucleotide into the genome. The introduction may be performed by, for example, through introduction of a vector into the cell, the vector including a polynucleotide encoding a target polypeptide, and then, replicating the vector in the cell or integrating the polynucleotide into the genome.

The introduction of the gene may be performed by any method known in the art, for example, transformation, transfection, and electroporation.

As used herein, the term “vehicle” or “vector” refers to a nucleic acid molecule that is able to deliver other nucleic acids linked thereto. Examples of the vector are a plasmid vector or a virus-derived vector, or the like. A plasmid is a double-stranded circular DNA molecule linkable with another DNA. Examples of the vector are a plasmid expression vector, a virus expression vector, or a combination thereof.

As used herein, the gene engineering may be performed by a molecular biological method known in the art.

The term “parent cell” as used herein refers to an original cell, for example, a non-genetically engineered cell of the same type with respect to an engineered microorganism. Regarding a particular genetic modification, the “parent cell” may be a cell that lacks the particular genetic modification. Thus, the parent cell may be a cell that is used as a starting material to produce a genetically engineered microorganism having increased activity of a given protein (for example, a protein having a sequence identity of about 85% or more to gluokinase). Thus, for example, a microorganism that has been genetically modified to increase the activity of glucokinase in the microorganism can be produced from a parent cell that does not contain the genetic modification that increases the activity of the glucokinase. The same comparison can be applied to other genetic modifications.

The terms “gene” and “polynucleotide” as used herein are synonymous and refer to a nucleic acid fragment that encodes a particular protein, and may optionally include a regulatory sequence of a 5′-non coding sequence and/or a 3′-non coding sequence.

The term “sequence identity” of a nucleic acid or polypeptide refers to the degree of identity between bases or amino acid residues of two corresponding sequences obtained after the sequences are aligned so as to best match in certain comparable regions. The sequence identity is a value that is obtained by comparing two sequences in certain comparable regions via optimal alignment of the two sequences, in which portions of the sequences in the certain comparable regions may be added or deleted compared to reference sequences. A percentage of the sequence identity may be calculated by, for example, comparing two optimally aligned sequences in the entire comparable regions, determining the number of locations in which the same amino acids or nucleic acids appear to obtain the number of matching locations, dividing the number of matching locations by the total number of locations in the comparable regions (e.g., the size of a range), and multiplying a result of the division by 100 to obtain the percentage of the sequence identity. The percentage of the sequence identity may be determined using a known sequence comparison program, for example, BLASTN (NCBI), BLASTP (NCBI), CLC Main Workbench (CLC bio), and MegAlign™ (DNASTAR Inc).

The term “genetic modification” as used herein refers to an artificial modification of a constitution or structure of the genetic material of a cell.

An aspect of the disclosure provides a recombinant microorganism including a genetic modification that increases activity of glucokinase. The recombinant microorganism may then have enhanced cellulose productivity.

In one embodiment, the genetic modification may be a modification that increases expression of a gene or polypeptide encoding the glucokinase. The genetic modification may be an increase in the copy number of a gene or polypeptide encoding the glucokinase, or a modification of a regulatory sequence of expression of a gene or polypeptide encoding the glucokinase.

The glucokinase (glk) may catalyze the conversion of ATP+glucose to ADP+glucose-6-phosphate, and may also catalyze the reverse reaction. The glucokinase may belong to EC 2.7.1.2. The glucokinase may be a polypeptide having sequence identity of 85% or more (e.g., 90% or more, 95% or more, or 98% or 99% or more) to the amino acid sequence of SEQ ID NO: 2, 4, or 6. In some embodiments, the polypeptide may be derived from Escherichia coli, Saccharomyces cerevisiae, or Zymomonas mobilis. The glucokinase may have the nucleotide sequence of SEQ ID NO: 1, 3, or 5.

In certain embodiments, the recombinant microorganism may further include one or more genetic modifications selected from a genetic modification that increases activity of glucose permease, a genetic modification that increases activity of phosphoglucomutase (PGM), and a genetic modification that increases activity of UTP-glucose pyrophosphorylase (UGP).

The genetic modifications that increase the activity of glucose permease, that increase the activity of phosphoglucomutase (PGM), and that increase the activity of UTP-glucose pyrophosphorylase (UGP) may be to increase expression of a gene or polynucleotide encoding each enzyme, or may be to increase the copy number of a gene or polynucleotide encoding each enzyme, or may be to modify a regulatory sequence of expression of a gene polynucleotide encoding each enzyme, or some combination thereof

The glucose permease may have activity of catalyzing the transport of glucose from outside a cell into a cell. The glucose permease may be a polypeptide having sequence identity of 85% or more (e.g., 90% or more, 95% or more, or 98% or 99% or more) to the amino acid sequence of SEQ ID NO: 8. The polypeptide may be a product of E. coli galP. A gene or polynucleotide encoding the polypeptide may have the nucleotide sequence of SEQ ID NO: 7.

The PGM may catalyze the transfer of phosphate at the 1′-position of glucose to the 6′-position of the same glucose or to an opposite direction from the 6′-position. The PGM may belong to EC 5.4.2.2. The PGM may be a polypeptide having a sequence identity of about 85% or more (e.g., 90% or more, 95% or more, or 98% or 99% or more) to the amino acid sequence of SEQ ID: 10, 12 or 14. The gene or polynucleotide encoding the polypeptide may have the nucleotide sequence of SEQ ID NO: 9, 11, or 13.

The UGP may belong to EC 2.7.7.9. The UGP may catalyze the following reaction:

Glucose-1-phosphate+UTP↔UDP-glucose+pyrophosphate.

The UGP may be a polypeptide having a sequence identity of about 85% or more (e.g., 90% or more, 95% or more, or 98% or 99% or more) to the amino acid sequence of each of SEQ ID: 16, 18, or 20. A gene or polynucleotide encoding the polypeptide may have the nucleotide sequence of SEQ ID NO: 15, 17, or 19. The polypeptide of SEQ ID NO: 16, 18, or 20 may be a product of the E. coli galU gene, the M. tuberculosis UGP gene, or the X. campestris UGP gene.

In certain embodiments, the microorganism may further include a genetic modification that increases expression of a glcP gene; a PGM gene; an UGP gene; a glcP gene and a PGM gene; a glcP gene and an UGP gene; a PGM gene and an UGP gene; a glcP gene and a PGM gene; and/or an UGP gene. The genetic modification may be introduction of a gene or polynucleotide encoding glk, glcP, PGM, UGP, or a combination thereof. One or more of the genes or polynucleotides encoding glk, glcP, PGM, UGP, or a combination thereof introduced into the microorganism may be present in a chromosome (integrated into a chromosome) or outside a chromosome (not within a chromosome). Furthermore, the recombinant microorganism may comprise a plurality of any one or more of the genes or polynucleotides encoding the enzymes, for example, 2 or more, 5 or more, 10 or more, 30 or more, 50 or more, 100 or more, or 1,000 or more.

The microorganism may belong to a genus Komagataeibacter, a genus Acetobacter, a genus Gluconacetobacter, or a genus Enterobacter, each of which has bacterial cellulose productivity. The microorganism may belong to the genus Komagataeibacter and may have bacterial cellulose productivity. The microorganism may be K. xylinus (also referred to as “G. xylinus”), K. rhaeticus, K. swingsii, K. kombuchae, K. nataicola, or K. sucrofermentans.

Another aspect provides a method of producing cellulose, the method including culturing a recombinant microorganism including a genetic modification that is to increase activity of glucokinase, in a medium to produce cellulose. The method can further comprise collecting the cellulose from a culture.

All aspects of the recombinant microorganism used in the method are as described above.

The culturing may be performed in a medium containing a carbon source, such as glucose. The medium used for culturing the microorganism may be any general culture medium that is suitable for growth of a host cell, such as a minimal or complex medium containing proper supplements. The suitable medium may be commercially available or prepared by prepared by a preparation method known in the art.

The culture medium may satisfy the requirements of a particular microorganism according to a product selected for the culturing. The medium may contain a component selected from the group consisting of a carbon source, a nitrogen source, a salt, a trace element, and a combination thereof.

The culturing conditions may be appropriately controlled for production of a selected product, for example, cellulose. The culturing may be performed under aerobic conditions for cell proliferation. The culturing may be performed by spinner culture or static culture without shaking. The concentration of the microorganism may be at a level which ensures that there is sufficient space so that secretion of cellulose is not inhibited.

The term “culture conditions” as used herein refers to conditions for culturing the microorganism. Such culture conditions may include, for example, a carbon source, a nitrogen source, or an oxygen condition utilized by the microorganism. The carbon source that may be utilized by the microorganism may include a monosaccharides, a disaccharide, or a polysaccharide. The carbon source may include, as an assimilable sugar, glucose, fructose, mannose, or galactose. The nitrogen source may be an organic nitrogen compound or an inorganic nitrogen compound. The nitrogen source may be exemplified by amino acid, amide, amine, nitrate, or ammonium salt. The oxygen condition for culturing the microorganism may be an aerobic condition of a normal oxygen partial pressure or a low-oxygen condition including about 0.1% to about 10% of oxygen in the atmosphere. A metabolic pathway may be modified in accordance with a carbon source or a nitrogen source that may be actually used by a microorganism.

The medium may include ethanol or cellulose, The ethanol may be used in an amount in a range of about 0.1 to about 5% (v/v), for example, about 0.3 to about 2.5% (v/v), about 0.3 to about 2.0% (v/v), about 0.3 to about 1.5% (v/v), about 0.3 to about 1.25% (v/v), about 0.3 to about 1.0% (v/v), about 0.3 to about 0.7% (v/v), or about 0.5 to about 3.0% (v/v), with respect to a volume of the medium. The cellulose may be used in an amount in a range of about 0.5 to about 5% (w/v), about 0.5 to about 2.5% (w/v), about 0.5 to about 1.5% (w/v), or about 0.7 to about 1.25% (w/v), with respect to a weight of the medium. The cellulose may be carboxylated cellulose. The cellulose may be carboxymethyl cellulose (CMC), and the CMC may be sodium CMC.

The method may include collecting the cellulose from the culture. The collection method may be for example, collecting of a cellulose pellicle formed on the top of the medium. The cellulose pellicle may be collected by physically separating or stripping off the cellulose pellicle from the medium, or by removing the medium from the culture leaving the pellicle. The method of collection may involve collecting of the cellulose pellicle while maintaining the shape of the pellicle without significant damage.

Another aspect provides a method of producing the microorganism having enhanced cellulose productivity, the method including introducing a gene or polynucleotide encoding glucokinase into a microorganism having cellulose productivity.

The introducing of the gene may be accomplished by introducing a vector including the gene or polynucleotide into the microorganism. The genetic modification also may include amplification of the gene, manipulation of a regulatory sequence of the gene, or manipulation of a sequence of the gene itself. The manipulation may be an insertion, a substitution, a conversion, or the addition of a nucleotide.

The method of producing the microorganism, according to certain embodiments, may further include introducing a glcP gene, a PGM gene, an UGP gene, or a combination thereof, into the microorganism.

The recombinant microorganism according to an aspect of the present invention may be used to produce cellulose in a high yield.

The method of producing cellulose according to another aspect of the present invention may be used to efficiently produce cellulose.

The method of producing the microorganism having enhanced cellulose productivity according to another aspect of the present invention may be used to efficiently produce the microorganism having enhanced cellulose productivity.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Hereinafter, the present invention will be described in more detail with reference to Examples. However, these Examples are for illustrative purposes only, and the scope of the present invention is not intended to be limited by these Examples.

### Example 1. Preparation of Komagataeibacter xylinus Including Glucose Permease, GLK1, PGM, and UGP Genes and Production of Cellulose

In this Example, glucose was used as a carbon source and four genes, i.e., glucose permease, GLK1, PGM, and UGP genes that act on a cellulose synthesis pathway, were selected. The selected four genes were each or in combination introduced into K. xylinus (Korean Culture Center of Microorganisms, KCCM 41431) and GDH gene-depleted K. xylinus, thereby examining effects of the gene introduction on cellulose productivity.

(1) Preparation of GDH Gene-Deleted K. xylinus

A membrane-bound pyrroloquinoline-quinone (PQQ)-dependent glucose dehydrogenase (GDH) gene in K. xylinus (Korean Culture Center of Microorganisms, KCCM 41431) was inactivated by homologous recombination. A specific procedure is as follows.

To delete the GDH gene by homologous recombination, fragments of the 5′- and 3′-ends of the GDH gene were obtained by PCR amplification using a genomic sequence of K. xylinus as a template and a set of primers of GDH-5-F (SEQ ID NO: 21) and GHD-5-R (SEQ ID NO: 22) and a set of primers of GDH-3-F (SEQ ID NO: 23) and GHD-3-R (SEQ ID NO: 24). Further, a neo gene (nptII) fragment which is a kanamycin resistance gene derived from Tn5 was obtained by PCR amplification using a set of primers of SEQ ID NOs: 25 and 26. Three of the fragments of the 5′- and 3′-ends of the GDH gene and the kanamycin resistance gene fragment were cloned into SacI and XbaI restriction sites of a pGEM-3zf vector (#P2271, Promega Corp.) using an 1n-fusion HD cloning kit (#PT5162-1, Clontech) to prepare pGz-dGDH. This vector thus obtained was transformed into K. xylinus by electroporation. The transformed K. xylinus strain was spread on a HS-agar medium (0.5% peptone, 0.5% yeast extract, 0.27% Na₂HPO₄, 0.15% citric acid, 2% glucose, and 1.5% bacto-agar) supplemented with 100 μg/ml of kanamycin, and then cultured at 30° C. A strain having a kanamycin resistance was selected to confirm deletion of the GDH gene. The GDH gene deletion was confirmed, and this strain was designated as K. xylinus (Δgdh).

(2) Introduction of Glucose Permease Gene

E. coli galP gene, i.e., a nucleotide sequence of SEQ ID NO: 7, was introduced into K. xylinus (Δgdh). E. coli galP is a galactose-proton symporter. E. coli galP is an integral membrane protein that facilitates the transport of cations together with import galactose and/or glucose in to the cell. A specific introduction procedure is as follows.

PCR was performed by using primers of Ec.galP-F (SEQ ID NOs: 17 and 18) and a genomic sequence of E. coli as a template, thereby obtaining a glucose permease gene derived from the microorganism.

Then, the glucose permease gene was cloned into the PstI restriction site of pCSa (SEQ ID NO: 29) using an In-fusion HD cloning kit (#PT5162-1, Clontech) to allow expression under the Tac promoter. The vector thus obtained was transformed into K. xylinus (Δgdh) by electroporation. The transformed K. xylinus (Δgdh) strain was spread on a HS-agar medium (0.5% peptone, 0.5% yeast extract, 0.27% Na₂HPO₄, 0.15% citric acid, 2% glucose, and 1.5% bacto-agar) supplemented with 100 μg/ml of chloramphenicol, and then cultured at 30° C. A strain having chloramphenicol resistance was selected to prepare glucose permease gene-overexpressing strains.

(3) Introduction of Glucokinase Gene

E. coli glk, S. cerevisiae glk, and Z. mobilis glk genes, i.e., nucleotide sequences of SEQ ID NOs: 1, 3, and 5, respectively, were introduced into K. xylinus (Δgdh). A specific introduction procedure is as follows.

PCR was performed by using 3 sets of primers, i.e., primers of SEQ ID NOs: 30 and 31; primers of SEQ ID NOs: 32 and 33; and primers of SEQ ID NOs: 34 and 35, and a genomic sequence of each of E. coli, S. cerevisiae, and Z. mobilis, respectively, as a template, thereby each obtaining a glk gene derived from the microorganism.

The glk gene was cloned into the PstI restriction site of pCSa (SEQ ID NO: 29) using an In-fusion HD cloning kit (#PT5162-1, Clontech) to allow expression under the Tac promoter. The vector thus obtained was transformed into K. xylinus (Δgdh) by electroporation. The transformed K. xylinus (Δgdh) strain was spread on an HS-agar medium (0.5% peptone, 0.5% yeast extract, 0.27% Na₂HPO₄, 0.15% citric acid, 2% glucose, and 1.5% bacto-agar) supplemented with 100 μg/ml of chloramphenicol, and then cultured at 30° C. Strains having chloramphenicol resistance were selected to prepare glk gene-overexpressing strains.

(4) Introduction of Phosphoglucomutase Gene

X. campestris xanA, B. subtilis pgca, and K. xylinus pgm genes, i.e., nucleotide sequences of SEQ ID NOs: 9, 11, and 13, respectively, were introduced into K. xylinus (Δgdh). A specific introduction procedure is as follows.

PCR was performed by using 3 sets of primers, i.e., primers of SEQ ID NOs: 36 and 37; primers of SEQ ID NOs: 38 and 39; and primers of SEQ ID NOs: 40 and 41, and a genomic sequence of each of X. campestris, B. subtilis, and K. xylinus, respectively, as a template, thereby each obtaining a pgm gene derived from the microorganism.

The pgm gene was cloned into the PstI restriction site of pCSa (SEQ ID NO: 29) using an In-fusion HD cloning kit (#PT5162-1, Clontech) to allow expression under the Tac promoter. The vector thus obtained was transformed into K. xylinus by electroporation. The transformed K. xylinus strain was spread on an HS-agar medium (0.5% peptone, 0.5% yeast extract, 0.27% Na₂HPO₄, 0.15% citric acid, 2% glucose, and 1.5% bacto-agar) supplemented with 100 μg/ml of chloramphenicol, and then cultured at 30° C. Strains having a chloramphenicol resistance were selected to prepare gpm gene-overexpressing strains.

(5) Introduction of UTP-Glucose Pyrophosphorylase Gene

E. coli galU, M. tuberculosis galU, or X. campestris ugp genes, i.e., nucleotide sequences of SEQ ID NO: 15, 42 (codon optimized), or 19, was introduced into K. xylinus (Δgdh). A specific introduction procedure is as follows.

PCR was performed by using 3 sets of primers, i.e., primers of SEQ ID NOs: 43 and 44; primers of SEQ ID NOs: 45 and 46; and primers of SEQ ID NOs: 47 and 48, and a genomic sequence of E. coli, a M. tuberculosis ugp gene codon-optimized for expression in K. xylinus, or a genomic sequence of X. campestris, respectively, as a template, thereby each obtaining a UTP-glucose pyrophosphorylase gene.

The UTP-glucose pyrophosphorylase gene was cloned into the PstI restriction site of pCSa (SEQ ID NO: 29) using an In-fusion HD cloning kit (#PT5162-1, Clontech) to allow expression under the Tac promoter. The vector thus obtained was transformed into K. xylinus by electroporation. The transformed K. xylinus strain was spread on an HS-agar medium (0.5% peptone, 0.5% yeast extract, 0.27% Na₂HPO₄, 0.15% citric acid, 2% glucose, and 1.5% bacto-agar) supplemented with 100 μg/ml of chloramphenicol, and then cultured at 30° C. Strains having a chloramphenicol resistance were selected to prepare ugp gene-overexpressing strains.

(6) Introduction of Glucokinase, Glucose Permease, Phosphoglucomutase, and UTP-Glucose Pyrophosphorylase Genes

In K. xylinus (Korean Culture Center of Microorganisms, KCCM 41431), four genes were introduced into a genome of K. xylinus by homologous recombination. The four genes are E. coli galP gene having activity of glucose permease, E. coli glk gene having activity of glucokinase, X. campestris xanA gene having activity of phosphoglucomutase, and E. coli galU gene having activity of UTP-glucose pyrophosphorylase. A specific introduction procedure is as follows.

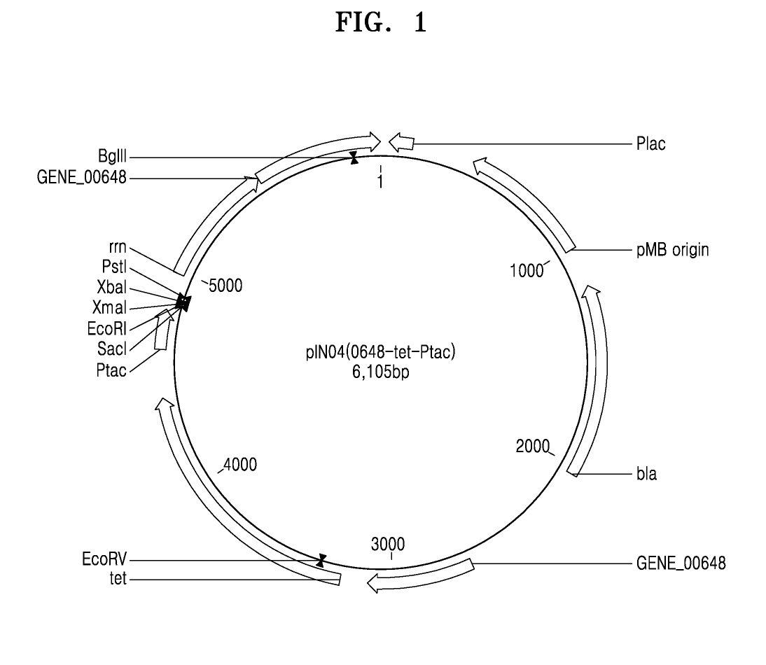

To prepare a vector for insertion into the genome of K. xylinus, fragments of the 5′- and 3′-ends of Gene 00648 were used for insertion into the genome of Gene 00648 by homologous recombination and a genomic sequence of K. xylinus was used as a template, thereby synthesizing a polynucleotide including a tetracycline resistance gene, a promoter, and a terminator. The synthesized polynucleotide was used as a template for PCR with a set of primers of SEQ ID NOs: 49 and 50. Gene fragments obtained by said PCR amplification were cloned into EcoRI and HindIII restriction sites of a pUC19 vector (#N3041S, NEB) using an In-fusion HD cloning kit (#PT5162-1, Clontech), thereby preparing a pIN04(0648-tet-Ptac) vector (SEQ ID NO: 54). FIG. 1 shows the pIN04 vector for use in insertion of four genes.

Four types of fragments of E. coli galP, E. coli glk, X. campestris xanA, and E. coli galU genes were synthesized and amplified by PCR using a set of primers of SEQ ID NOs: 51 and 52. Gene fragments obtained therefrom were cloned into SacI and XbaI restriction sites of the pIN04(0648-tet-Ptac) vector using an In-fusion HD cloning kit (#PT5162-1, Clontech), thereby preparing a pIN04-galP-xanA-galU-glk vector (SEQ ID NO: 53).

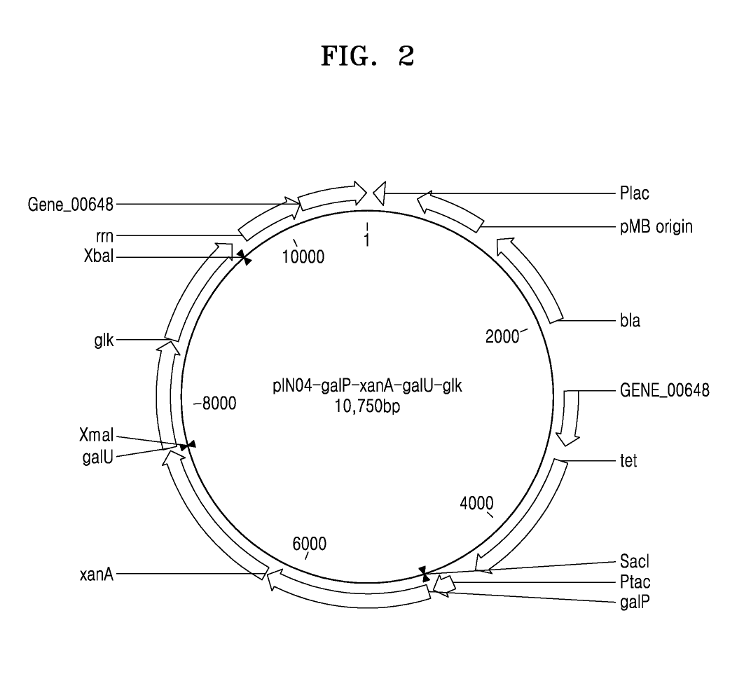

FIG. 2 shows the structure of the pIN04-galP-xanA-galU-glk vector including four genes. In FIG. 2, galP indicates E. coli galP gene, glk indicates E. coli glk gene, galU indicates E. coli galU gene, and xanA indicates X. campestris xanA gene.

The vector thus obtained was transformed into K. xylinus by electroporation. The transformed K. xylinus strain was spread on a HS-agar medium (0.5% peptone, 0.5% yeast extract, 0.27% Na₂HPO₄, 0.15% citric acid, 2% glucose, and 1.5% bacto-agar) supplemented with 100 μg/ml of tetracycline, and then cultured at 30° C. A strain having a tetracycline resistance was selected to confirm the insertion, and this strain was designated as K. xylinus (+4G).

(7) Confirmation of Cellulose Production Amount

The designated K. xylinus strains prepared in Examples 1(2) to (5) were inoculated into a 250-mL flask containing 25 ml of Hestrin and Schramm(HS) medium (0.5% peptone, 0.5% yeast extract, 0.27% Na₂HPO₄, 0.15% citric acid, and 2-4% glucose), respectively and cultured at 230 rpm at 30° C. for 5 days. Then, the product cellulose was quantified. In the case of the glucose permease-overexpressing recombinant strain, the glk gene-overexpressing recombinant strain, the pgm gene-overexpressing recombinant strain, and the ugp gene-overexpressing recombinant strain, 100 μg/ml of chloramphenicol was added to media. Glucose was analyzed by high performance liquid chromatography (HPLC) equipped with an Aminex HPX-87H column (Bio-Rad, USA), and the cellulose production amount was measured after washing the cellulose solid formed in the flask with 0.1 N sodium hydroxide and water and then drying the cellulose solid in an oven at 60° C. Table 1 shows the strain-dependent cellulose product amounts.

TABLE 1

| Strain | Cellulose production amount (mg/L) |
|---|---|
| Control group | 524.0 |
| Ec galP | 576.4 |
| Xc xanA | 807.0 |
| Ec galU | 979.88 |
| Zc glk | 602.6 |

Referring to Table 1, the control group is K. xylinus (Δgdh), Ec galP, Xc XanA, Ec galU, and Ec glk indicates the introduction of K. xylinus (Δgdh) E. coli galP, X. campestris xanA, E. coli galU, and E. coli glk genes, respectively. As shown in Table 1, each of the recombinant strains showed increased cellulose product amounts compared to that of the control group.

In addition, regarding K. xylinus (+4G) prepared in Example 1(6), the cellulose production amount was measured after culturing. The culturing was performed in the same manner as described above, except that a HSE medium containing 1.0 (v/v) % ethanol of the HE medium and a 250-mL flask containing 25 ml of the HSE medium were used and the culturing was performed for 6 days instead of 5 days. Then, the cellulose production amount was measured. The results are shown in Table 2.

TABLE 2

| Strain | Cellulose production amount (g/L) |
|---|---|
| Wild-type K. xylinus | 5.092 |
| K. xylinus (+4G) | 5.804 |

As shown in Table 2, K. xylinus (+4G) produced cellulose in a significantly increased amount as compared to the wild-type strain.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A genus Komagataeibacter recombinant microorganism comprising a genetic modification that increases glucokinase activity.

2. The recombinant microorganism of claim 1, wherein the genetic modification increases the expression of a gene encoding a glucokinase.

3. The recombinant microorganism of claim 2, wherein the genetic modification is an increase in the copy number of a gene encoding the glucokinase or a modification of a regulatory sequence to increase expression of a gene encoding the glucokinase.

4. The recombinant microorganism of claim 1, wherein the glucokinase belongs to EC 2.7.1.2.

5. The recombinant microorganism of claim 1, wherein the glucokinase catalyzes the conversion of ATP+glucose to ADP+glucose-6-phosphate, and catalyzes the reverse reaction.

6. The recombinant microorganism of claim 1, wherein the glucokinase is a polypeptide having sequence identity of 85% or more to the amino acid sequence of SEQ ID NO: 10, 12, or 14.

7. The recombinant microorganism of claim 1, wherein the microorganism further comprises one or more genetic modifications selected from a genetic modification that increases activity of glucose permease, a genetic modification that increases activity of phosphoglucomutase (PGM), and a genetic modification that increases activity of UTP-glucose pyrophosphorylase (UGP).

8. The recombinant microorganism of claim 1, wherein the microorganism further comprises one or more genetic modifications selected from a genetic modification that increases the expression of a gene encoding glucose permease, a genetic modification that increases the expression of a gene encoding phosphoglucomutase (PGM), and a genetic modification that increases the expression of a gene encoding UTP-glucose pyrophosphorylase (UGP).

9. The recombinant microorganism of claim 1, wherein the microorganism further comprises one or more genetic modifications selected from an increase in copy number of a gene encoding glucose permease, an increase in copy number of a gene encoding phosphoglucomutase (PGM), and an increase in copy number of a gene encoding UTP-glucose pyrophosphorylase (UGP);
or the microorganism further comprises one or more genetic modifications selected from a modification of a regulatory sequence that increases expression of a gene encoding glucose permease, expression of a gene encoding phosphoglucomutase (PGM), and expression of a gene encoding UTP-glucose pyrophosphorylase (UGP).

10. The recombinant microorganism of claim 7, wherein the glucose permease is an enzyme that transports glucose into a cell, and PGM and UGP each belong to EC 5.4.2.2 and EC 2.7.7.9.

11. The recombinant microorganism of claim 7, wherein the glucose permease has about 85% sequence identity to SEQ ID NO: 8; PGM has about 85% sequence identity to SEQ ID NO: 10, 12, or 14, and UGP has about 85% sequence identity to SEQ ID NO: 16, 18, or 20.

12. The recombinant microorganism of claim 1, wherein the microorganism is Komagataeibacter xylinus.

13. A method of producing cellulose, the method comprising:
culturing the recombinant microorganism of claim 1 in a culture medium to produce cellulose; and
collecting the cellulose from said culture medium.

14. The method of claim 13, wherein the recombinant microorganism comprises a genetic modification that increases expression of a gene encoding glucokinase.

15. The method of claim 14, wherein the genetic modification is an increase the copy number of a gene encoding the glucokinase, or a modification of a regulatory sequence that increases expression of a gene encoding the glucokinase.

16. The method of claim 14, wherein the recombinant microorganism further comprises one or more genetic modifications selected from a genetic modification that increases activity of glucose permease, a genetic modification that increases activity of phosphoglucomutase (PGM), and a genetic modification that increases activity of UTP-glucose pyrophosphorylase (UGP).

17. The method of claim 16, wherein the recombinant microorganism further comprises one or more genetic modifications selected from a genetic modification that increases the expression of a gene encoding glucose permease, a genetic modification that increases the expression of a gene encoding phosphoglucomutase (PGM), and a genetic modification that increases the expression of a gene encoding UTP-glucose pyrophosphorylase (UGP).

18. The method of claim 16, wherein the recombinant microorganism further comprises one or more genetic modifications selected from an increase in copy number of a gene encoding glucose permease, an increase in copy number of a gene encoding phosphoglucomutase (PGM), and an increase in copy number of a gene encoding UTP-glucose pyrophosphorylase (UGP);
or the recombinant microorganism further comprises one or more genetic modifications selected from a modification of a regulatory sequence that increases expression of a gene encoding glucose permease, expression of a gene encoding phosphoglucomutase (PGM), and expression of a gene encoding UTP-glucose pyrophosphorylase (UGP).

19. The method of claim 16, wherein the glucose permease transports glucose into the cell, and PGM and UGP each belong to EC 5.4.2.2 and EC 2.7.7.9.

20. A method of producing a recombinant microorganism having enhanced cellulose productivity, the method comprising:
introducing an exogenous gene encoding glucokinase into a microorganism to provide a recombinant microorganism having enhanced cellulose productivity.


SEQUENCE LISTING (54 sequences)

SEQ ID NO: 1 - DNA, 966 bp, Escherichia coli
atgacaaagtatgcattagtcggtgatgtgggcggcaccaacgcacgtcttgctctgtgtgatattgccagtggtgaaatctcgcaggctaagacctattcagggcttgattaccccagcctcgaagcggtcattcgcgtttatcttgaagaacataaggtcgaggtgaaagacggctgtattgccatcgcttgcccaattaccggtgactgggtggcgatgaccaaccatacctgggcgttctcaattgccgaaatgaaaaagaatctcggttttagccatctggaaattattaacgattttaccgctgtatcgatggcgaacccgatgctgaaaaaagagcatctgattcagtttggtggcgcagaaccggtcgaaggtaagcctattgcggtttacggtgccggaacggggcttggggttgcgcatctggtccatgtcgataagcgttgggtaagcttgccaggcgaaggcggtcacgttgattttgcgccgaatagtgaagaagaggccattatcctcgaaatattgcgtgcggaaattggtcatgtttcggcggaggcgtgcctttctggccctgggctggtgaatttgtatcgcgcaattgtgaaagctgacaaccgcctgccagaaaatctcaagccaaaagatattaccgaacgcgcgctggctgacagctgcaccgattgccgccgcgcattgtcgctgttttgcgtcattatgggccgttttggcggcaatctggcgctcaatctcgggacatttggcggcgtgtttattgcgggcggtatcgtgccgcgcttccttgagttcttcaaaggctccggtttccgtgccgcatttgaagataaagggcgctttaaagaatatgtccatgatattccggtgtatctcatcgtccatgacaatccgggccttctcggttccggtgcacatttacgccagaccttaggtcacattctgtaa

SEQ ID NO: 2 - protein, 321 aa, Escherichia coli
MTKYALVGDVGGTNARLALCDIASGEISQAKTYSGLDYPSLEAVIRVYLEEHKVEVKDGCIAIACPITGDWVAMTNHTWAFSIAEMKKNLGFSHLEIINDFTAVSMANPMLKKEHLIQFGGAEPVEGKPIAVYGAGTGLGVAHLVHVDKRWVSLPGEGGHVDFAPNSEEEAIILEILRAEIGHVSAEACLSGPGLVNLYRAIVKADNRLPENLKPKDITERALADSCTDCRRALSLFCVIMGRFGGNLALNLGTFGGVFIAGGIVPRFLEFFKGSGFRAAFEDKGRFKEYVHDIPVYLIVHDNPGLLGSGAHLRQTLGHIL

SEQ ID NO: 3 - DNA, 1503 bp, Saccharomyces cerevisiae
atgtcattcgacgacttacacaaagccactgagagagcggtcatccaggccgtggaccagatctgcgacgatttcgaggttacccccgagaagctggacgaattaactgcttacttcatcgaacaaatggaaaaaggtctagctccaccaaaggaaggccacacattggcctcggacaaaggtcttcctatgattccggcgttcgtcaccgggtcacccaacgggacggagcgcggtgttttactagccgccgacctgggtggtaccaatttccgtatatgttctgttaacttgcatggagatcatactttctccatggagcaaatgaagtccaagattcccgatgatttgctagacgatgagaacgtcacatctgacgacctgtttgggtttctagcacgtcgtacactggcctttatgaagaagtatcacccggacgagttggccaagggtaaagacgccaagcccatgaaactggggttcactttctcataccctgtagaccagacctctctaaactccgggacattgatccgttggaccaagggtttccgcatcgcggacaccgtcggaaaggatgtcgtgcaattgtaccaggagcaattaagcgctcagggtatgcctatgatcaaggttgttgcattaaccaacgacaccgtcggaacgtacctatcgcattgctacacgtccgataacacggactcaatgacgtccggagaaatctcggagccggtcatcggatgtattttcggtaccggtaccaatgggtgctatatggaggagatcaacaagatcacgaagttgccacaggagttgcgtgacaagttgataaaggagggtaagacacacatgatcatcaatgtcgaatgggggtccttcgataatgagctcaagcacttgcctactactaagtatgacgtcgtaattgaccagaaactgtcaacgaacccgggatttcacttgtttgaaaaacgtgtctcagggatgttcttgggtgaggtgttgcgtaacattttagtggacttgcactcgcaaggcttgcttttgcaacagtacaggtccaaggaacaacttcctcgccacttgactacacctttccagttgtcatccgaagtgctgtcgcatattgaaattgacgactcgacaggtctacgtgaaacagagttgtcattattacagagtctcagactgcccaccactccaacagagcgtgttcaaattcaaaaattggtgcgcgcgatttctaggagatctgcgtatttagccgccgtgccgcttgccgcgatattgatcaagacaaatgctttgaacaagagatatcatggtgaagtcgagatcggttgtgatggttccgttgtggaatactaccccggtttcagatctatgctgagacacgccttagccttgtcacccttgggtgccgagggtgagaggaaggtgcacttgaagattgccaaggatggttccggagtgggtgccgccttgtgtgcgcttgtagcatga

SEQ ID NO: 4 - protein, 500 aa, Saccharomyces cerevisiae
MSFDDLHKATERAVIQAVDQICDDFEVTPEKLDELTAYFIEQMEKGLAPPKEGHTLASDKGLPMIPAFVTGSPNGTERGVLLAADLGGTNFRICSVNLHGDHTFSMEQMKSKIPDDLLDDENVTSDDLFGFLARRTLAFMKKYHPDELAKGKDAKPMKLGFTFSYPVDQTSLNSGTLIRWTKGFRIADTVGKDVVQLYQEQLSAQGMPMIKVVALTNDTVGTYLSHCYTSDNTDSMTSGEISEPVIGCIFGTGTNGCYMEEINKITKLPQELRDKLIKEGKTHMIINVEWGSFDNELKHLPTTKYDVVIDQKLSTNPGFHLFEKRVSGMFLGEVLRNILVDLHSQGLLLQQYRSKEQLPRHLTTPFQLSSEVLSHIEIDDSTGLRETELSLLQSLRLPTTPTERVQIQKLVRAISRRSAYLAAVPLAAILIKTNALNKRYHGEVEIGCDGSVVEYYPGFRSMLRHALALSPLGAEGERKVHLKIAKDGSGVGAALCALVA

SEQ ID NO: 5 - DNA, 984 bp, Zymomonas mobilis
atggaaattgttgcgattgacatcggtggaacgcatgcgcgtttctctattgcggaagtaagcaatggtcgggttctttctcttggagaagaaacaacttttaaaacggcagaacatgctagcttgcagttagcttgggaacgtttcggtgaaaaactgggtcgtcctctgccacgtgccgcagctattgcatgggctggcccggttcatggtgaagttttaaaacttaccaataacccttgggtattaagaccagctactctgaatgaaaagctggacatcgatacgcatgttctgatcaatgacttcggcgcggttgcccacgcggttgcgcatatggattcttcttatctggatcatatttgtggtcctgatgaagcgcttcctagcgatggtgttatcactattcttggtccgggaacgggcttgggtgttgcccatctgttgcggactgaaggccgttatttcgtcatcgaaactgaaggcggtcatatcgactttgctccgcttgacagacttgaagacaaaattctggcacgtttacgtgaacgtttccgccgcgtttctatcgaacgcattatttctggcccgggtcttggtaatatctacgaagcactggctgccattgaaggcgttccgttcagcttgctggatgatattaaattatggcagatggctttggaaggtaaagacaaccttgctgaagccgctttggatcgcttctgcttgagccttggcgctatcgctggtgatcttgctttggcacagggtcgaaccagtgttgttattggcggtggtgtcggtcttcgtatcgcttcccatttgccagaatctggtttccgtcagcgctttgtttcaaaaggacgctttgaacgcgtcatgtccaagattccggttaagttgattacttatccgcagcctggactgttgggtgcgcagctgcctatgccaacaaatattctgaagttgaataatattttttaa

SEQ ID NO: 6 - protein, 327 aa, Zymomonas mobilis
MEIVAIDIGGTHARFSIAEVSNGRVLSLGEETTFKTAEHASLQLAWERFGEKLGRPLPRAAAIAWAGPVHGEVLKLTNNPWVLRPATLNEKLDIDTHVLINDFGAVAHAVAHMDSSYLDHICGPDEALPSDGVITILGPGTGLGVAHLLRTEGRYFVIETEGGHIDFAPLDRLEDKILARLRERFRRVSIERIISGPGLGNIYEALAAIEGVPFSLLDDIKLWQMALEGKDNLAEAALDRFCLSLGAIAGDLALAQGRTSVVIGGGVGLRIASHLPESGFRQRFVSKGRFERVMSKIPVKLITYPQPGLLGAQLPMPTNILKLNNIF

SEQ ID NO: 7 - DNA, 1395 bp, Escherichia coli
atgcctgacgctaaaaaacaggggcggtcaaacaaggcaatgacgtttttcgtctgcttccttgccgctctggcgggattactctttggcctggatatcggtgtaattgctggcgcactgccgtttattgcagatgaattccagattacttcgcacacgcaagaatgggtcgtaagctccatgatgttcggtgcggcagtcggtgcggtgggcagcggctggctctcctttaaactcgggcgcaaaaagagcctgatgatcggcgcaattttgtttgttgccggttcgctgttctctgcggctgcgccaaacgttgaagtactgattctttcccgcgttctactggggctggcggtgggtgtggcctcttataccgcaccgctgtacctctctgaaattgcgccggaaaaaattcgtggcagtatgatctcgatgtatcagttgatgatcactatcgggatcctcggtgcttatctttctgataccgccttcagctacaccggtgcatggcgctggatgctgggtgtgattatcatcccggcaattttgctgctgattggtgtcttcttcctgccagacagcccacgttggtttgccgccaaacgccgttttgttgatgccgaacgcgtgctgctacgcctgcgtgacaccagcgcggaagcgaaacgcgaactggatgaaatccgtgaaagtttgcaggttaaacagagtggctgggcgctgtttaaagagaacagcaacttccgccgcgcggtgttccttggcgtactgttgcaggtaatgcagcaattcaccgggatgaacgtcatcatgtattacgcgccgaaaatcttcgaactggcgggttataccaacactaccgagcaaatgtgggggaccgtgattgtcggcctgaccaacgtacttgccacctttatcgcaatcggccttgttgaccgctggggacgtaaaccaacgctaacgctgggcttcctggtgatggctgctggcatgggcgtactcggtacaatgatgcatatcggtattcactctccgtcggcgcagtatttcgccatcgccatgctgctgatgtttattgtcggttttgccatgagtgccggtccgctgatttgggtactgtgctccgaaattcagccgctgaaaggccgcgattttggcatcacctgctccactgccaccaactggattgccaacatgatcgttggcgcaacgttcctgaccatgctcaacacgctgggtaacgccaacaccttctgggtgtatgcggctctgaacgtactgtttatcctgctgacattgtggctggtaccggaaaccaaacacgtttcgctggaacatattgaacgtaatctgatgaaaggtcgtaaactgcgcgaaataggcgctcacgattaa

SEQ ID NO: 8 - protein, 464 aa, Escherichia coli
MPDAKKQGRSNKAMTFFVCFLAALAGLLFGLDIGVIAGALPFIADEFQITSHTQEWVVSSMMFGAAVGAVGSGWLSFKLGRKKSLMIGAILFVAGSLFSAAAPNVEVLILSRVLLGLAVGVASYTAPLYLSEIAPEKIRGSMISMYQLMITIGILGAYLSDTAFSYTGAWRWMLGVIIIPAILLLIGVFFLPDSPRWFAAKRRFVDAERVLLRLRDTSAEAKRELDEIRESLQVKQSGWALFKENSNFRRAVFLGVLLQVMQQFTGMNVIMYYAPKIFELAGYTNTTEQMWGTVIVGLTNVLATFIAIGLVDRWGRKPTLTLGFLVMAAGMGVLGTMMHIGIHSPSAQYFAIAMLLMFIVGFAMSAGPLIWVLCSEIQPLKGRDFGITCSTATNWIANMIVGATFLTMLNTLGNANTFWVYAALNVLFILLTLWLVPETKHVSLEHIERNLMKGRKLREIGAHD

SEQ ID NO: 9 - DNA, 1347 bp, Xanthomonas campestris
atgacgctacccgccttcaaggcctacgatattcgcggccgcgtgccggatgaactcaacgaggacttggcccgccgcatcggcgtggcactggcggcgcagctggatcaagggcccgtggtcctgggccacgatgtgcgcctggcgagcccggcactgcaggaagccctgtctgccggcctgcgtgccagcggccgcgaggtgatcgacatcggcctgtgtggcaccgaggaggtctatttccagaccgatcacctcaaggccgccggcggcgtgatggtcaccgccagccacaacccgatggactacaacggcatgaagctggtgcgtgaacaggcgcgaccgatcagctccgataccggcctgttcgccatccgcgacacggtcgcggccgacactgctgctgcaggcgagcccaccgctgccgagcacagccgcaccgacaagaccgcgtatctggagcacctgctcagctacgtggaccgcagcacgctcaagccgctcaagctggtggtcaacgccggcaacggcggcgccggcctgatcgtcgacctgctggcaccgcatctgccattcgaattcgtgcgcgtcttccacgagcccgatggcaacttccccaacggcatccccaacccgctgctgcaggaaaaccgcgacgccaccgccaaggcggtcaaggaacacggcgccgacttcgggattgcctgggatggcgacttcgatcgttgcttcttcttcgatcacactggccgcttcatcgagggctattacctggtcggcctgctggcgcaagccatcctggccaagcagcccggcggcaaggtcgtgcacgacccgcgcctgacctggaacacggtggagatggtggaagacgccggcggcattccggtgctgtgcaagagtggccacgccttcattaaggaaaagatgcgcagcgagaacgccgtctatggtggcgaaatgagcgcgcaccattacttccgcgaattcgcctacgccgactcgggcatgattccatggctgctgatcgccgagctggtctcgcaatcgggccgttcgctggcggacctggtcgaagcgcgcatgcagaagttcccatgcagcggcgagatcaacttcaaggtcgacgacgccaaggctgcggtcgcacgcgtcatggcgcattacggtgatcagtcaccggagctggattacaccgacggcatcagcgccgacttcgggcaatggcgcttcaacctgcgcagctccaacaccgagccgctgctgcgtctgaacgtggaaacgcgcggcgatgctgcactgctggagacgcgtacgcaggaaatttccaacctgttacgcggctga

SEQ ID NO: 10 - protein, 448 aa, Xanthomonas campestris
MTLPAFKAYDIRGRVPDELNEDLARRIGVALAAQLDQGPVVLGHDVRLASPALQEALSAGLRASGREVIDIGLCGTEEVYFQTDHLKAAGGVMVTASHNPMDYNGMKLVREQARPISSDTGLFAIRDTVAADTAAAGEPTAAEHSRTDKTAYLEHLLSYVDRSTLKPLKLVVNAGNGGAGLIVDLLAPHLPFEFVRVFHEPDGNFPNGIPNPLLQENRDATAKAVKEHGADFGIAWDGDFDRCFFFDHTGRFIEGYYLVGLLAQAILAKQPGGKVVHDPRLTWNTVEMVEDAGGIPVLCKSGHAFIKEKMRSENAVYGGEMSAHHYFREFAYADSGMIPWLLIAELVSQSGRSLADLVEARMQKFPCSGEINFKVDDAKAAVARVMAHYGDQSPELDYTDGISADFGQWRFNLRSSNTEPLLRLNVETRGDAALLETRTQEISNLLRG

SEQ ID NO: 11 - DNA, 1746 bp, Bacillus subtilis
atgacttggagaaagagctatgaacgctggaaacagacagaacatttagatctggaattaaaagagcgccttattgaattagagggagatgaacaggcccttgaggactgtttctataaagaccttgaattcggtaccggcggaatgcgcggggaaatcggcgccgggacaaatcggatgaatatttacactgtgcgcaaagcatcggccgggtttgcggcatacatctcgaagcaaggtgaggaagcgaaaaaacggggcgttgtcattgcttatgattcccgccataagtctccggagttcgcgatggaagcggcaaaaacacttgcgacacaaggcatccaaacatacgtgtttgatgagcttcgcccgacgccagagctgtcattcgctgttagacagctgaacgcttatggtggaattgtggtaacggcaagccataacccgcctgaatataacggctacaaagtatacggggatgatggcggccagctgcctccaaaggaagcggacatcgtcattgagcaggtaaacgcgattgaaaatgagctgacgatcacagtggacgaagaaaataagttaaaagaaaaaggcttaatcaaaatcatcggtgaagatattgataaagtttatacagaaaaactgacgtccatttctgtacatcctgaattatcggaagaagtagatgtaaaggttgttttcacaccgctgcatggaactgcaaataaaccggtcagacgcggtcttgaagcactcggctacaaaaatgtaacggttgtcaaagaacaggaactgccggattcaaacttctccactgttacatcgccgaacccggaagagcatgcggcattcgaatatgccattaagcttggggaggagcagaatgcagatattctcatcgcgacagatcctgatgctgaccgcctcggcatcgcggtgaaaaacgatcaaggcaaatatacagtgctgacaggaaaccaaaccggagcgttgctgcttcattacctgctttctgaaaagaaaaaacaaggcatcctgcctgataacggtgttgttctcaaaacgatcgtcacaagcgaaatcggccgtgctgtagcttcttcattcggccttgatacgattgatacgctgacaggctttaagtttatcggtgaaaagattaaggaatacgaagcatcaggccagtataccttccaattcggttatgaagagagctacggttatttaatcggggattttgcccgcgataaggacgccattcaggctgcgcttttggcagttgaagtttgcgcgttctataaaaaacagggaatgtcattgtatgaggcgctcatcaatctctttaacgaatatggtttttatcgtgaagggctgaaatccctgacgctgaaaggcaaacaaggagcagagcaaattgaagcgattcttgcttccttcagacaaaatccgccgcagaaaatggcgggcaaacaggttgtcacagcagaagattacgctgtaagcaaacggacgcttctgactgaaagcaaagaagaagccatcgacttgccaaaatcaaatgtattgaaatacttcctggaagacgggtcttggttctgtctccgtccttctggaactgagccgaaggttaaattttatttcgccgtaaaagggtcatctttggaagacagtgaaaagcgacttgccgtcctttctgaagatgtaatgaagacggtggatgaaattgttgagtcaacagcaaaataa

SEQ ID NO: 12 - protein, 581 aa, Bacillus subtilis
MTWRKSYERWKQTEHLDLELKERLIELEGDEQALEDCFYKDLEFGTGGMRGEIGAGTNRMNIYTVRKASAGFAAYISKQGEEAKKRGVVIAYDSRHKSPEFAMEAAKTLATQGIQTYVFDELRPTPELSFAVRQLNAYGGIVVTASHNPPEYNGYKVYGDDGGQLPPKEADIVIEQVNAIENELTITVDEENKLKEKGLIKIIGEDIDKVYTEKLTSISVHPELSEEVDVKVVFTPLHGTANKPVRRGLEALGYKNVTVVKEQELPDSNFSTVTSPNPEEHAAFEYAIKLGEEQNADILIATDPDADRLGIAVKNDQGKYTVLTGNQTGALLLHYLLSEKKKQGILPDNGVVLKTIVTSEIGRAVASSFGLDTIDTLTGFKFIGEKIKEYEASGQYTFQFGYEESYGYLIGDFARDKDAIQAALLAVEVCAFYKKQGMSLYEALINLFNEYGFYREGLKSLTLKGKQGAEQIEAILASFRQNPPQKMAGKQVVTAEDYAVSKRTLLTESKEEAIDLPKSNVLKYFLEDGSWFCLRPSGTEPKVKFYFAVKGSSLEDSEKRLAVLSEDVMKTVDEIVESTAK

SEQ ID NO: 13 - DNA, 1668 bp, Komagataeibacter xylinus
atgcccagcataagcccatttgccggcaagccggtcgatccggaccgtcttgtcaatatcgacgccctgcttgacgcctattacacccgcaagcccgaccccgccattgcaacgcagcgcgtggcgtttggcacgtcggggcaccgtggttcctcgctgaccaccagcttcaacgaaaaccacatcctgtcgatcagccaggcgattgccgactaccgcaagggcgcgggcataaccgggccgctgttcatcggcattgacacccatgcgctttcccgtcccgcgctgaaatccgcgctggaagtgttcgcggccaatggcgtggaagtccgcatcgatgcgagggacggctatacccccacgccggtcatctcgcacgcgatcctgacctataaccgcgaccgcagcacggaccttgccgatggcgtggtgatcaccccgtcgcataacccgccggaagatggcggctacaagtacaatcccccccatggtggcccggcggataccgacatcaccaaagtggtggaaaatgcggccaatgactacatggccaaaaagatggaaggcgtgaagcgtgtcagctttgaggatgcgctgaaggcccccaccaccaggcgtcatgattacatcacgccgtatgtggatgatctggccgccgtggtggacatggacgtgatccgtgaatccggcgtgtcgatcggcattgacccgctgggcggggccgcggtggattactggcagccgatcatcgacaaatacggcatcaacgccacgatcgtcagcaaggaagtggacccgaccttccgcttcatgaccgccgactgggacgggcagatccgcatggactgttcctccccctacgccatggcgcgccttgtcgggatgaaggacaaattcgacatcgccttcgccaatgataccgatgccgaccgccatggcatcgtgtcgggcaaatacgggcttatgaaccccaaccactatctggccgtcgcgattgaatacctgttcaacaaccgcgaaaactggaacgccagcgcgggcgtgggcaagacggtggtcagcagcagcatgatcgatcgcgtggccagggaaatcggccgcaagctggtggaagtgccggtcgggttcaagtggtttgtcgatgggctgtacaacggcacgctgggctttggcggggaagaaagtgctggcgcgtccttcctgcgccgtgccggcacggtgtggagcacggacaaggacggtatcatccttggcctgctggcagccgaaatcacggcccgcaccaggcgcacccccggtgctgcgtatgaggacatgaccaaacgcctgggcacgccgtactatgcgcggatcgacgccccggccgacccggaacagaaggccatcctgaaaaacctctcgcccgagcagatcggcatgaccgaactggcgggtgagccgatcctcagcaccctgaccaatgcaccgggcaacggggcggccattggcgggctgaaggtttcggcaaaggatggctggtttgccgcacgcccctcgggcacggaaaacgtctacaagatctacgccgaaagcttcaagagcgaggcgcacctcaaggccatccagaccgaggcgcaggatgcgatttccgccctgtttgccaaggccgccaagaaaaaggctggctga

SEQ ID NO: 14 - protein, 555 aa, Komagataeibacter xylinus
MPSISPFAGKPVDPDRLVNIDALLDAYYTRKPDPAIATQRVAFGTSGHRGSSLTTSFNENHILSISQAIADYRKGAGITGPLFIGIDTHALSRPALKSALEVFAANGVEVRIDARDGYTPTPVISHAILTYNRDRSTDLADGVVITPSHNPPEDGGYKYNPPHGGPADTDITKVVENAANDYMAKKMEGVKRVSFEDALKAPTTRRHDYITPYVDDLAAVVDMDVIRESGVSIGIDPLGGAAVDYWQPIIDKYGINATIVSKEVDPTFRFMTADWDGQIRMDCSSPYAMARLVGMKDKFDIAFANDTDADRHGIVSGKYGLMNPNHYLAVAIEYLFNNRENWNASAGVGKTVVSSSMIDRVAREIGRKLVEVPVGFKWFVDGLYNGTLGFGGEESAGASFLRRAGTVWSTDKDGIILGLLAAEITARTRRTPGAAYEDMTKRLGTPYYARIDAPADPEQKAILKNLSPEQIGMTELAGEPILSTLTNAPGNGAAIGGLKVSAKDGWFAARPSGTENVYKIYAESFKSEAHLKAIQTEAQDAISALFAKAAKKKAG

SEQ ID NO: 15 - DNA, 909 bp, Escherichia coli
atggctgccattaatacgaaagtcaaaaaagccgttatccccgttgcgggattaggaaccaggatgttgccggcgacgaaagccatcccgaaagagatgctgccacttgtcgataagccattaattcaatacgtcgtgaatgaatgtattgcggctggcattactgaaattgtgctggttacacactcatctaaaaactctattgaaaaccactttgataccagttttgaactggaagcaatgctggaaaaacgtgtaaaacgtcaactgcttgatgaagtgcagtctatttgtccaccgcacgtgactattatgcaagttcgtcagggtctggcgaaaggcctgggacacgcggtattgtgtgctcacccggtagtgggtgatgaaccggtagctgttattttgcctgatgttattctggatgaatatgaatccgatttgtcacaggataacctggcagagatgatccgccgctttgatgaaacgggtcatagccagatcatggttgaaccggttgctgatgtgaccgcatatggcgttgtggattgcaaaggcgttgaattagcgccgggtgaaagcgtaccgatggttggtgtggtagaaaaaccgaaagcggatgttgcgccgtctaatctcgctattgtgggtcgttacgtacttagcgcggatatttggccgttgctggcaaaaacccctccgggagctggtgatgaaattcagctcaccgacgcaattgatatgctgatcgaaaaagaaacggtggaagcctatcatatgaaagggaagagccatgactgcggtaataaattaggttacatgcaggccttcgttgaatacggtattcgtcataacacccttggcacggaatttaaagcctggcttgaagaagagatgggcattaagaagtaa

SEQ ID NO: 16 - protein, 302 aa, Escherichia coli
MAAINTKVKKAVIPVAGLGTRMLPATKAIPKEMLPLVDKPLIQYVVNECIAAGITEIVLVTHSSKNSIENHFDTSFELEAMLEKRVKRQLLDEVQSICPPHVTIMQVRQGLAKGLGHAVLCAHPVVGDEPVAVILPDVILDEYESDLSQDNLAEMIRRFDETGHSQIMVEPVADVTAYGVVDCKGVELAPGESVPMVGVVEKPKADVAPSNLAIVGRYVLSADIWPLLAKTPPGAGDEIQLTDAIDMLIEKETVEAYHMKGKSHDCGNKLGYMQAFVEYGIRHNTLGTEFKAWLEEEMGIKK

SEQ ID NO: 17 - DNA, 921 bp, Mycobacterium tuberculosis
atgagccgcccggaggtgctgacgcctttcacggccatcgtgcctgccgcgggtctgggtacgcgtttcctgcctgcgaccaagacggtgcccaaggagctgctgcccgtggtcgacacccccggtatcgagctggtggcagcagaggcagccgcggcgggtgccgaacgtcttgttatcgtcacctccgagggtaaggacggcgtggtcgcccacttcgtggaggacctggtgctcgagggcacgctggaggcccgtggcaagatcgccatgctggccaaggtgcgtcgcgccccggccctgatcaaggtcgaatccgtggtgcaggccgaaccgctggggctgggccatgccatcggctgtgtggaaccgacgctgtcgcccgacgaagacgcggtcgcggtgctgctgccggacgacctggtgctgccgaccggcgtcctggagacgatgtcgaaggtgcgcgccagccggggcggcaccgtgctgtgcgcgatcgaggtggcgcgcgaggaaatcagcgcctacggggtttttgatgtcgaaccggtccccgatggcgactacaccgacgatcccaacgtgctgaaggtccggggcatggtcgaaaagcccaaagccgaaacggcgccgtcgcggtatgcggcagcagggcggtacgttcttgaccgcgcgatcttcgatgcgctccgccgcattgaccagggggccggcggggaagtgcagctcaccgatgcgattgcgctgctgattgcggaaggccatcccgttcatgtggtcgtccaccaggggtcccgccatgacctgggcaatccgggcgggtatctcaaagcggcggttgattttgcccttgatcgcgatgattatggcccggatctccggcgctggctcgtggcgcgccttgggcttaccgaacagtga

SEQ ID NO: 18 - protein, 306 aa, Mycobacterium tuberculosis
MSRPEVLTPFTAIVPAAGLGTRFLPATKTVPKELLPVVDTPGIELVAAEAAAAGAERLVIVTSEGKDGVVAHFVEDLVLEGTLEARGKIAMLAKVRRAPALIKVESVVQAEPLGLGHAIGCVEPTLSPDEDAVAVLLPDDLVLPTGVLETMSKVRASRGGTVLCAIEVAREEISAYGVFDVEPVPDGDYTDDPNVLKVRGMVEKPKAETAPSRYAAAGRYVLDRAIFDALRRIDQGAGGEVQLTDAIALLIAEGHPVHVVVHQGSRHDLGNPGGYLKAAVDFALDRDDYGPDLRRWLVARLGLTEQ

SEQ ID NO: 19 - DNA, 894 bp, Xanthomonas campestris
atgagcaagcgtattcgcaaggccgtattccccgtcgcaggtcttgggacccgctttcttccggcaaccaagacggtgccgaaggaaatgctgccgatcatcgataaaccgcttatccagtacgccgtggacgaagccatccaggccggttgcgatacgctgatcttcgtcaccaatcgctacaagcactcgatcgccgactacttcgacaaggcctatgagctggagcagaagctcgagcgcgcaggcaagctcgagcaattggagctggtgcgccatgcactgcccgaaggcgtccgcgccatttttgtgacgcaggccgaagcgctcggcctgggccatgcggtgctgtgtgccaaggccgtggtcggtgacgaaccgttcgcagtgctgctgcccgacgacctgatgtggaaccgcggcgatgcggcgctgacccagatggccgatgtggcagaagcctccggcggcagcgtgatcgccgtggaagatgtgccgcacgacaagacggccagttatggcatcgtgtccaccgatgcgttcgacggccgcaagggccgcatcaatgccatcgtcgaaaagcccaagccggaagtggcgccgagcaatctggcggtcgtcggtcgttacgtgctgagcccgaagatcttcgagtacctggagtccacgggcgccggtgcaggtggcgagatccagctcaccgatgcgatcgccgaactgctcaagcaggaacaggtcgatgcgttccgtttcgagggccgtcgcttcgactgcggcgcgcacatcggtttgatcgaggccacggtgcactttgcgctcgagcacgagaagcatggcgcaccggcgaaggaaatcattcgcagcgccttggccgcggccgacgcacgcggctga

SEQ ID NO: 20 - protein, 297 aa, Xanthomonas campestris
MSKRIRKAVFPVAGLGTRFLPATKTVPKEMLPIIDKPLIQYAVDEAIQAGCDTLIFVTNRYKHSIADYFDKAYELEQKLERAGKLEQLELVRHALPEGVRAIFVTQAEALGLGHAVLCAKAVVGDEPFAVLLPDDLMWNRGDAALTQMADVAEASGGSVIAVEDVPHDKTASYGIVSTDAFDGRKGRINAIVEKPKPEVAPSNLAVVGRYVLSPKIFEYLESTGAGAGGEIQLTDAIAELLKQEQVDAFRFEGRRFDCGAHIGLIEATVHFALEHEKHGAPAKEIIRSALAAADARG

SEQ ID NO: 21 - DNA, 35 bp, Artificial Sequence
  Synthetic GDH-5-F primer
tagaatactcaagcttggagctaccagaccgtcca

SEQ ID NO: 22 - DNA, 33 bp, Artificial Sequence
  Synthetic GHD-5-R primer
tcagaccccgtagaacaaacatgccaaggttgc

SEQ ID NO: 23 - DNA, 33 bp, Artificial Sequence
  Synthetic primer
caacaccttcttcacttgaatggggtggccttg

SEQ ID NO: 24 - DNA, 35 bp, Artificial Sequence
  Synthetic primer
tatagggcgaattcgggcaggcggtcctgccacag

SEQ ID NO: 25 - DNA, 34 bp, Artificial Sequence
  Synthetic primer
tcacgccgccttcgcgtgaagaaggtgttgctga

SEQ ID NO: 26 - DNA, 33 bp, Artificial Sequence
  Synthetic primer
aacaccagcgtgcccttctacggggtctgacgc

SEQ ID NO: 27 - DNA, 36 bp, Artificial Sequence
  Synthetic primer
tagagtcgacctgcaatgcctgacgctaaaaaacag

SEQ ID NO: 28 - DNA, 36 bp, Artificial Sequence
  Synthetic primer
ccaagcttgcatgccttaatcgtgagcgcctatttc

SEQ ID NO: 29 - DNA, 3128 bp, Artificial Sequence
  Synthetic pCSa vector
gaattcagccagcaagacagcgatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatcacctgtaagtcggacgcgatgcgtccggcgtagaggatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacagggacgagctattgattgggtaccgagctcgaattcgtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagcatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaaaggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacatagatctcccgggtaccgagctctctagaaagaaggagggacgagctattgatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttttttaaggcagttattggtgcccttaaacgcctggttgctacgcctgaataagtgataataagcggatgaatggcagaaattc

SEQ ID NO: 30 - DNA, 36 bp, Artificial Sequence
  Synthetic pCSa vector
tagagtcgacctgcaatgacaaagtatgcattagtc

SEQ ID NO: 31 - DNA, 36 bp, Artificial Sequence
  Synthetic primer
ccaagcttgcatgccttacagaatgtgacctaaggt

SEQ ID NO: 32 - DNA, 36 bp, Artificial Sequence
  Synthetic primer
tagagtcgacctgcaatgtcattcgacgacttacac

SEQ ID NO: 33 - DNA, 36 bp, Artificial Sequence
  Synthetic primer
ccaagcttgcatgcctcatgctacaagcgcacacaa

SEQ ID NO: 34 - DNA, 36 bp, Artificial Sequence
  Synthetic primer
tagagtcgacctgcaatggaaattgttgcgattgac

SEQ ID NO: 35 - DNA, 36 bp, Artificial Sequence
  Synthetic primer
ccaagcttgcatgccttaaaaaatattattcaactt

SEQ ID NO: 36 - DNA, 36 bp, Artificial Sequence
  Synthetic primer
tagagtcgacctgcaatgacgctacccgccttcaag

SEQ ID NO: 37 - DNA, 36 bp, Artificial Sequence
  Synthetic primer
ccaagcttgcatgcctcagccgcgtaacaggttgga

SEQ ID NO: 38 - DNA, 36 bp, Artificial Sequence
  Synthetic primer
tagagtcgacctgcaatgacttggagaaagagctat

SEQ ID NO: 39 - DNA, 36 bp, Artificial Sequence
  Synthetic primer
ccaagcttgcatgccttattttgctgttgactcaac

SEQ ID NO: 40 - DNA, 36 bp, Artificial Sequence
  Synthetic primer
tagagtcgacctgcaatgcccagcataagcccattt

SEQ ID NO: 41 - DNA, 36 bp, Artificial Sequence
  Synthetic primer
ccaagcttgcatgcctcagccagcctttttcttggc

SEQ ID NO: 42 - DNA, 921 bp, Artificial Sequence; identical to SEQ ID NO: 17
  Synthetic Mycobacterium tuberculosis ugp

SEQ ID NO: 43 - DNA, 34 bp, Artificial Sequence
  Synthetic primer
tagagtcgacctgcacaaaacacgaacagtccag

SEQ ID NO: 44 - DNA, 35 bp, Artificial Sequence
  Synthetic primer
ccaagcttgcatgccgctataaaaaaacggcgtcg

SEQ ID NO: 45 - DNA, 36 bp, Artificial Sequence
  Synthetic primer
tagagtcgacctgcaatgagccgcccggaggtgctg

SEQ ID NO: 46 - DNA, 36 bp, Artificial Sequence
  Synthetic primer
ccaagcttgcatgcctcactgttcggtaagcccaag

SEQ ID NO: 47 - DNA, 33 bp, Artificial Sequence
  Synthetic primer
tagagtcgacctgcaatacggtcgttccattcc

SEQ ID NO: 48 - DNA, 33 bp, Artificial Sequence
  Synthetic primer
ccaagcttgcatgccggacccaaaacgacgaac

SEQ ID NO: 49 - DNA, 39 bp, Artificial Sequence
  Synthetic primer
gacggccagtgaatttggcaccatacattccggtttccc

SEQ ID NO: 50 - DNA, 39 bp, Artificial Sequence
  Synthetic primer
tgattacgccaagctaatatggcagccatcaataccccg

SEQ ID NO: 51 - DNA, 42 bp, Artificial Sequence
  Synthetic primer
attgattgggtaccgagctcgagctattgatgcctgacgcta

SEQ ID NO: 52 - DNA, 42 bp, Artificial Sequence
  Synthetic primer
gcctgcaggtcgactctagattacagaatgtgacctaaggtc

SEQ ID NO: 53 - DNA, 10750 bp, Artificial Sequence
  Synthetic PIN04-galP-xanA-galU-glk
agcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaatttggcaccatacattccggtttccccagctattccgctgatggccagcagatcgtgtaccgggtgtggggggaaaacaacatgggcctgcgcatactggacctgcggacccggcagacacgcgtgctgaccacggcatatgacaatctgcccggctggtcgcccgatggcagccggatcgtgttcacgcgccgggtcgatgacgaaaattatgatattttcaccatccgtcccgacggaaccgggctgaagcggctgaccaccagcggcgccaatgacggccatgccgtatggaccgccgatggccgcatcatgtggtcaagcggcgtatatggcttccgtgacgaagcggccctgtatgacaatacgttccagccttacgggcagatattcatcatgaatgccgacggcagtcacaatcggatcctgacggacagcctgtgggaggattccatgcccctttatgtcccggcatgatgtcgcgatcatgcacgataacgggcaaggcatggtcttatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatttttaaggcagttattggtgcccgcaaccatctcgggcttctggatttccgatcacctgtaagtcggacgcgatgcgtccggcgtagaggatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacagggacgagctattgattgggtaccgagctcgagctattgatgcctgacgctaaaaaacaggggcggtcaaacaaggcaatgacgtttttcgtctgcttccttgccgctctggcgggattactctttggcctggatatcggtgtaattgctggcgcactgccgtttattgcagatgaattccagattacttcgcacacgcaagaatgggtcgtaagctccatgatgttcggtgcggcagtcggtgcggtgggcagcggctggctctcctttaaactcgggcgcaaaaagagcctgatgatcggcgcaattttgtttgttgccggttcgctgttctctgcggctgcgccaaacgttgaagtactgattctttcccgcgttctactggggctggcggtgggtgtggcctcttataccgcaccgctgtacctctctgaaattgcgccggaaaaaattcgtggcagtatgatctcgatgtatcagttgatgatcactatcgggatcctcggtgcttatctttctgataccgccttcagctacaccggtgcatggcgctggatgctgggtgtgattatcatcccggcaattttgctgctgattggtgtcttcttcctgccagacagcccacgttggtttgccgccaaacgccgttttgttgatgccgaacgcgtgctgctacgcctgcgtgacaccagcgcggaagcgaaacgcgaactggatgaaatccgtgaaagtttgcaggttaaacagagtggctgggcgctgtttaaagagaacagcaacttccgccgcgcggtgttccttggcgtactgttgcaggtaatgcagcaattcaccgggatgaacgtcatcatgtattacgcgccgaaaatcttcgaactggcgggttataccaacactaccgagcaaatgtgggggaccgtgattgtcggcctgaccaacgtacttgccacctttatcgcaatcggccttgttgaccgctggggacgtaaaccaacgctaacgctgggcttcctggtgatggctgctggcatgggcgtactcggtacaatgatgcatatcggtattcactctccgtcggcgcagtatttcgccatcgccatgctgctgatgtttattgtcggttttgccatgagtgccggtccgctgatttgggtactgtgctccgaaattcagccgctgaaaggccgcgattttggcatcacctgctccactgccaccaactggattgccaacatgatcgttggcgcaacgttcctgaccatgctcaacacgctgggtaacgccaacaccttctgggtgtatgcggctctgaacgtactgtttatcctgctgacattgtggctggtaccggaaaccaaacacgtttcgctggaacatattgaacgtaatctgatgaaaggtcgtaaactgcgcgaaataggcgctcacgattaacccgagctattgatgacgctacccgccttcaaggcctacgatattcgcggccgcgtgccggatgaactcaacgaggacttggcccgccgcatcggcgtggcactggcggcgcagctggatcaagggcccgtggtcctgggccacgatgtgcgcctggcgagcccggcactgcaggaagccctgtctgccggcctgcgtgccagcggccgcgaggtgatcgacatcggcctgtgtggcaccgaggaggtctatttccagaccgatcacctcaaggccgccggcggcgtgatggtcaccgccagccacaacccgatggactacaacggcatgaagctggtgcgtgaacaggcgcgaccgatcagctccgataccggcctgttcgccatccgcgacacggtcgcggccgacactgctgctgcaggcgagcccaccgctgccgagcacagccgcaccgacaagaccgcgtatctggagcacctgctcagctacgtggaccgcagcacgctcaagccgctcaagctggtggtcaacgccggcaacggcggcgccggcctgatcgtcgacctgctggcaccgcatctgccattcgaattcgtgcgcgtcttccacgagcccgatggcaacttccccaacggcatccccaacccgctgctgcaggaaaaccgcgacgccaccgccaaggcggtcaaggaacacggcgccgacttcgggattgcctgggatggcgacttcgatcgttgcttcttcttcgatcacactggccgcttcatcgagggctattacctggtcggcctgctggcgcaagccatcctggccaagcagcccggcggcaaggtcgtgcacgacccgcgcctgacctggaacacggtggagatggtggaagacgccggcggcattccggtgctgtgcaagagtggccacgccttcattaaggaaaagatgcgcagcgagaacgccgtctatggtggcgaaatgagcgcgcaccattacttccgcgaattcgcctacgccgactcgggcatgattccatggctgctgatcgccgagctggtctcgcaatcgggccgttcgctggcggacctggtcgaagcgcgcatgcagaagttcccatgcagcggcgagatcaacttcaaggtcgacgacgccaaggctgcggtcgcacgcgtcatggcgcattacggtgatcagtcaccggagctggattacaccgacggcatcagcgccgacttcgggcaatggcgcttcaacctgcgcagctccaacaccgagccgctgctgcgtctgaacgtggaaacgcgcggcgatgctgcactgctggagacgcgtacgcaggaaatttccaacctgttacgcggctgacccggggagctattgatggctgccattaatacgaaagtcaaaaaagccgttatccccgttgcgggattaggaaccaggatgttgccggcgacgaaagccatcccgaaagagatgctgccacttgtcgataagccattaattcaatacgtcgtgaatgaatgtattgcggctggcattactgaaattgtgctggttacacactcatctaaaaactctattgaaaaccactttgataccagttttgaactggaagcaatgctggaaaaacgtgtaaaacgtcaactgcttgatgaagtgcagtctatttgtccaccgcacgtgactattatgcaagttcgtcagggtctggcgaaaggcctgggacacgcggtattgtgtgctcacccggtagtgggtgatgaaccggtagctgttattttgcctgatgttattctggatgaatatgaatccgatttgtcacaggataacctggcagagatgatccgccgctttgatgaaacgggtcatagccagatcatggttgaaccggttgctgatgtgaccgcatatggcgttgtggattgcaaaggcgttgaattagcgccgggtgaaagcgtaccgatggttggtgtggtagaaaaaccgaaagcggatgttgcgccgtctaatctcgctattgtgggtcgttacgtacttagcgcggatatttggccgttgctggcaaaaacccctccgggagctggtgatgaaattcagctcaccgacgcaattgatatgctgatcgaaaaagaaacggtggaagcctatcatatgaaagggaagagccatgactgcggtaataaattaggttacatgcaggccttcgttgaatacggtattcgtcataacacccttggcacggaatttaaagcctggcttgaagaagagatgggcattaagaagtaaggggagctattgatgacaaagtatgcattagtcggtgatgtgggcggcaccaacgcacgtcttgctctgtgtgatattgccagtggtgaaatctcgcaggctaagacctattcagggcttgattaccccagcctcgaagcggtcattcgcgtttatcttgaagaacataaggtcgaggtgaaagacggctgtattgccatcgcttgcccaattaccggtgactgggtggcgatgaccaaccatacctgggcgttctcaattgccgaaatgaaaaagaatctcggttttagccatctggaaattattaacgattttaccgctgtatcgatggcgaacccgatgctgaaaaaagagcatctgattcagtttggtggcgcagaaccggtcgaaggtaagcctattgcggtttacggtgccggaacggggcttggggttgcgcatctggtccatgtcgataagcgttgggtaagcttgccaggcgaaggcggtcacgttgattttgcgccgaatagtgaagaagaggccattatcctcgaaatattgcgtgcggaaattggtcatgtttcggcggaggcgtgcctttctggccctgggctggtgaatttgtatcgcgcaattgtgaaagctgacaaccgcctgccagaaaatctcaagccaaaagatattaccgaacgcgcgctggctgacagctgcaccgattgccgccgcgcattgtcgctgttttgcgtcattatgggccgttttggcggcaatctggcgctcaatctcgggacatttggcggcgtgtttattgcgggcggtatcgtgccgcgcttccttgagttcttcaaaggctccggtttccgtgccgcatttgaagataaagggcgctttaaagaatatgtccatgatattccggtgtatctcatcgtccatgacaatccgggccttctcggttccggtgcacatttacgccagaccttaggtcacattctgtaatctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacggatggcctttttgccttaggcatccgttcgcacgatacctgccgcgacaccgccgacagacaggcacgttgcgattaacacgcctggcacagccgtgcggcactgatccgtacctttcgattacccgcagcttgctggcaagttgaaaccagaaatattgattagataatataatatatcataatttatttatcatcatggtatatagaaatgtaatttaaattcatggaacatgaattgatacaatttgcttcaaatatcgcgttgacacaactcagccgctaacatagaagccccaactattgggagatgaaaatgaagcaaatattacaaaaattcattatgaaacgacctccacaaaataattccgaatatgttcataaaataaatgattatacaatatttatagtttgtatgatggcatttttttcatgcaccattcttaccttatttcaaataagatctatcgattacaatatagaaattttcttaaattcattcgcaagaaaaagcaagtttgtgaatgcactcctttcatccctgacatatgatagtatgaccgtcggggtattgatggctgccatatt

SEQ ID NO: 54 - DNA, 6105 bp, Artificial Sequence
  Synthetic PIN04(648-tet-Ptac)
agcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaatttggcaccatacattccggtttccccagctattccgctgatggccagcagatcgtgtaccgggtgtggggggaaaacaacatgggcctgcgcatactggacctgcggacccggcagacacgcgtgctgaccacggcatatgacaatctgcccggctggtcgcccgatggcagccggatcgtgttcacgcgccgggtcgatgacgaaaattatgatattttcaccatccgtcccgacggaaccgggctgaagcggctgaccaccagcggcgccaatgacggccatgccgtatggaccgccgatggccgcatcatgtggtcaagcggcgtatatggcttccgtgacgaagcggccctgtatgacaatacgttccagccttacgggcagatattcatcatgaatgccgacggcagtcacaatcggatcctgacggacagcctgtgggaggattccatgcccctttatgtcccggcatgatgtcgcgatcatgcacgataacgggcaaggcatggtcttatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatttttaaggcagttattggtgcccgcaaccatctcgggcttctggatttccgatcacctgtaagtcggacgcgatgcgtccggcgtagaggatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacagggacgagctattgattgggtaccgagctcgaattcgtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacggatggcctttttgccttaggcatccgttcgcacgatacctgccgcgacaccgccgacagacaggcacgttgcgattaacacgcctggcacagccgtgcggcactgatccgtacctttcgattacccgcagcttgctggcaagttgaaaccagaaatattgattagataatataatatatcataatttatttatcatcatggtatatagaaatgtaatttaaattcatggaacatgaattgatacaatttgcttcaaatatcgcgttgacacaactcagccgctaacatagaagccccaactattgggagatgaaaatgaagcaaatattacaaaaattcattatgaaacgacctccacaaaataattccgaatatgttcataaaataaatgattatacaatatttatagtttgtatgatggcatttttttcatgcaccattcttaccttatttcaaataagatctatcgattacaatatagaaattttcttaaattcattcgcaagaaaaagcaagtttgtgaatgcactcctttcatccctgacatatgatagtatgaccgtcggggtattgatggctgccatatt